(19) United States
(12) Patent Application Publication
(10) Pub. No.: US 2025/0354191 A1
(43) Pub. Date: Nov. 20, 2025

(54) SYSTEM AND METHOD FOR SELF-ADMINISTERING A CULTURE TEST

(71) Applicant: Gadi PORATH, Kiriat Tivon (IL)

(72) Inventor: Gadi PORATH, Kiriat Tivon (IL)

(21) Appl. No.: 19/284,737

(22) Filed: Jul. 30, 2025

Related U.S. Application Data
(63) Continuation-in-part of application No. 18/432,082, filed on Feb. 5, 2024.
(60) Provisional application No. 63/448,288, filed on Feb. 26, 2023.

(51) Int. Cl. C12Q 1/04; C12Q 1/06

(52) U.S. Cl. CPC C12Q 1/045; C12Q 1/06

(57) ABSTRACT

Disclosed herein is a system for self-administering an infection culture test, the system including a housing having a cover and a compartment for storing a bacterial culture and to facilitate bacterial growth, wherein the compartment includes a pad having a partitioning element attached to a film, wherein the partitioning element separates a dry chromogenic medium into regions, wherein the medium is coated with a growth medium and a chromogenic substrate, and a transparent cover to hermetically seal the compartment, and one or more smart devices configured to record one or more images of the bacteria within the apparatus and provide a determination whether an infection is present and to identify what specific bacteria is present.

### CROSS-REFERENCE TO RELATED APPLICATIONS

This application is continuation-in-part from U.S. patent application Ser. No. 18/432,082, filed Feb. 5, 2024, which claims the benefit of priority to U.S. Provisional Patent Application No. 63/448,288, filed Feb. 26, 2023, entitled “SYSTEM AND METHOD FOR SELF-ADMINISTRATING A CULTURE TEST”. The entirety of the aforementioned application is incorporated herein by reference.

### BACKGROUND OF THE INVENTION

The invention relates to the field of bacteria culture testing.

A urinary tract infection (“UTI) is an infection of the urinary system, which includes the kidneys, ureters, bladder and urethra.

Streptococcal pharyngitis, also known as streptococcal sore throat (strep throat), is pharyngitis (an infection of the pharynx, the back of the throat) caused by Streptococcus pyogenes, a gram-positive, group A streptococcus.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

There is provided, in accordance with an embodiment, an apparatus for self-administering an infection culture test, the apparatus including a housing having a cover and a compartment for storing a bacterial culture and to facilitate bacterial growth, wherein the compartment includes a pad having a partitioning element attached to a film, wherein the partitioning element separates a dry chromogenic medium into regions, wherein the medium is coated with a growth medium and a chromogenic substrate, and a transparent cover to hermetically seal the compartment.

In some embodiments, the apparatus further includes a heating device, wherein the heating element is attachable to the housing to facilitate providing a predetermined temperature to expedite growth of the bacteria during the predetermined incubation period.

In some embodiments, the apparatus further includes ventilation ports.

In some embodiments, the housing further includes an antibiotics compartment to facilitate exposing the bacteria to antibiotics during the predetermined incubation period.

In some embodiments, the apparatus further includes an antibiotics compartment having a plurality of compartments for storing at least one type of antibiotic effective for treatment of the bacteria.

In some embodiments, the apparatus further includes a color scheme and basic colors to facilitate calibrating a camera configured to record at least one image thereby providing a visual comparison for determining whether there is a bacterial infection and to identify the specific bacterial culture.

In some embodiments, the window cover is connected to the housing with hinges to facilitate opening and closing the cover.

In some embodiments, the window cover is configured to slide onto the compartment.

In some embodiments, the window cover is clear to enable visually monitoring the bacterial growth during the predetermined incubation period.

In some embodiments, the apparatus further includes a carbon dioxide generator configured to generate carbon dioxide thereby expediting culture growth.

In some embodiments, the carbon dioxide generator is positioned beneath the filter.

In some embodiments, the carbon dioxide generator includes a tub for storing sodium bicarbonate.

In some embodiments, the tub is connected to the compartment to facilitate mixing fluids from compartment with the sodium bicarbonate to produce the carbon dioxide.

There is further provided, in accordance with an embodiment, a system for self-administering an infection culture test, the system including a housing having a cover and a compartment for storing a bacterial culture and to facilitate bacterial growth, wherein the compartment includes a pad having a partitioning element attached to a film, wherein the partitioning element separates a dry chromogenic medium into regions, wherein the medium is coated with a growth medium and a chromogenic substrate, and a transparent cover to hermetically seal the compartment, and one or more smart devices configured to record one or more images of the bacteria within the apparatus and provide a determination whether an infection is present and to identify what specific bacteria is present.

In some embodiments, the one or more smart devices include one or more cameras for recording the one or more images, one or more processors configured to analyze the one or more image to determine whether an infection is present by identify the specific bacteria, and one or more communication interfaces configured to provide the one or more images and determination of infection to a medical practitioner for assistance.

In some embodiments, the one or more smart devices further include a memory for storing the images and determination of infection.

In some embodiments, the apparatus further includes a color scheme and basic colors to facilitate calibrating the at least one camera configured to record at least one image thereby providing a visual comparison for determining whether there is a bacterial infection and to identify the specific bacterial culture.

In some embodiments, the system further includes a specimen cup for collecting a fluid sample, a swab for taking a specimen sample from the fluid sample, and a vial including a nutrition liquid broth, wherein the specimen sample is inserted into the vial to create a bacterial mixture for placing in the housing.

In some embodiments, the swab includes hydrophobic fibers operative to collect a sample within a range of 1-5 microliters.

In some embodiments, the system further includes a heating device, the heating device is attachable to the housing to facilitate providing a predetermined temperature to expedite growth of the bacteria.

In some embodiments, the apparatus further includes ventilation ports.

In some embodiments, the housing further includes an antibiotics compartment to facilitate exposing the bacteria to antibiotics.

In some embodiments, the apparatus further includes an antibiotics compartment having a plurality of compartments for storing at least one type of antibiotic to which the bacteria is exposed while the bacteria is within housing.

In some embodiments, the window cover is connected to the housing with hinges to facilitate opening and closing the cover.

In some embodiments, the window cover is configured to slide onto the compartment.

In some embodiments, the window cover is clear to enable visually monitoring the bacterial growth.

In some embodiments, the apparatus further includes a carbon dioxide generator configured to generate carbon dioxide thereby expediting culture growth.

In some embodiments, the carbon dioxide generator includes a tub for storing sodium bicarbonate.

In some embodiments, the carbon dioxide generator is positioned beneath the filter.

In some embodiments, the tub is connected to the compartment to facilitate mixing fluids from compartment with the sodium bicarbonate to produce the carbon dioxide.

### BRIEF DESCRIPTION OF DRAWINGS

Exemplary embodiments are illustrated in referenced figures. Dimensions of components and features shown in the figures are generally chosen for convenience and clarity of presentation and are not necessarily shown to scale. The figures are listed below.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described. References to previously presented elements are implied without necessarily further citing the drawing or description in which they appear.

FIG. 1 is a schematic illustration of a system for self-administering a culture test, according to certain exemplary embodiment;

FIGS. 2A-2B are schematic illustrations of an apparatus of the system of FIG. 1 for self-administering the culture test, according to certain exemplary embodiments;

FIG. 3 is a schematic illustration of the apparatus of FIGS. 2A-2B including a cover, according to certain exemplary embodiment;

FIGS. 4A-4C are schematic illustrations of the apparatus of FIGS. 2A-2B including a sliding cover, according to certain exemplary embodiment;

FIG. 5 is a schematic illustration of a housing of the apparatus of FIGS. 2A-2B, according to certain exemplary embodiment;

FIG. 6 is a schematic illustration of a filter of the apparatus of FIGS. 2A-2B, according to certain exemplary embodiment;

FIG. 7 is a schematic illustration of a housing for the apparatus of FIGS. 2A-2B, according to certain exemplary embodiment;

FIG. 8 is a schematic illustration of an internal electric heating element and thermostat during incubation for the apparatus of FIGS. 2A-2B, according to certain exemplary embodiment;

FIG. 9 is a side view schematic illustration of the apparatus of FIGS. 2A-2B, according to certain exemplary embodiment;

FIG. 10 is a schematic illustration of an antibiotic compartment for the apparatus of FIGS. 2A-2B, according to certain exemplary embodiment;

FIG. 11 is a schematic illustration of the housing of FIG. 7 including the antibiotic compartment of FIG. 10, according to certain exemplary embodiments;

FIG. 12 is a schematic illustration of a smart device of the system of FIG. 1, according to certain exemplary embodiment;

FIG. 13 is a schematic illustration of the apparatus of FIG. 1 including a carbon dioxide generator, according to certain exemplary embodiments;

FIG. 14 is a schematic illustration of another embodiment of a system for self-administering a culture test, according to certain embodiments;

FIG. 15 is a schematic illustration of an apparatus of the system of FIG. 14 for self-administering the culture test, according to certain exemplary embodiments;

FIGS. 16A-16C are schematic illustrations of a pad of the apparatus of FIG. 14, according to certain exemplary embodiments;

FIG. 17 is a schematic illustration of growth cultures on the pad of FIGS. 16A-16B, according to certain exemplary embodiments; and,

FIG. 18 is a schematic illustration of a swab of system of FIG. 14, according to certain exemplary embodiments.

### DETAILED DESCRIPTION OF THE INVENTION

Disclosed herein is a system for self-administering a culture test, according to certain exemplary embodiments. One skilled in the art will appreciate that the system, generally referred with reference number 100, includes a self-administered culture testing apparatus (“apparatus”), generally referred to with reference number 105, which is configured to facilitate culture testing based on fluids collected from a subject. It will be appreciated by one skilled in the art that the embodiments, disclosed herein relate to culture testing for urinary tract infections and Streptococcus infections, however this does not limit the infections which may be detected using the system and apparatus disclosed herein.

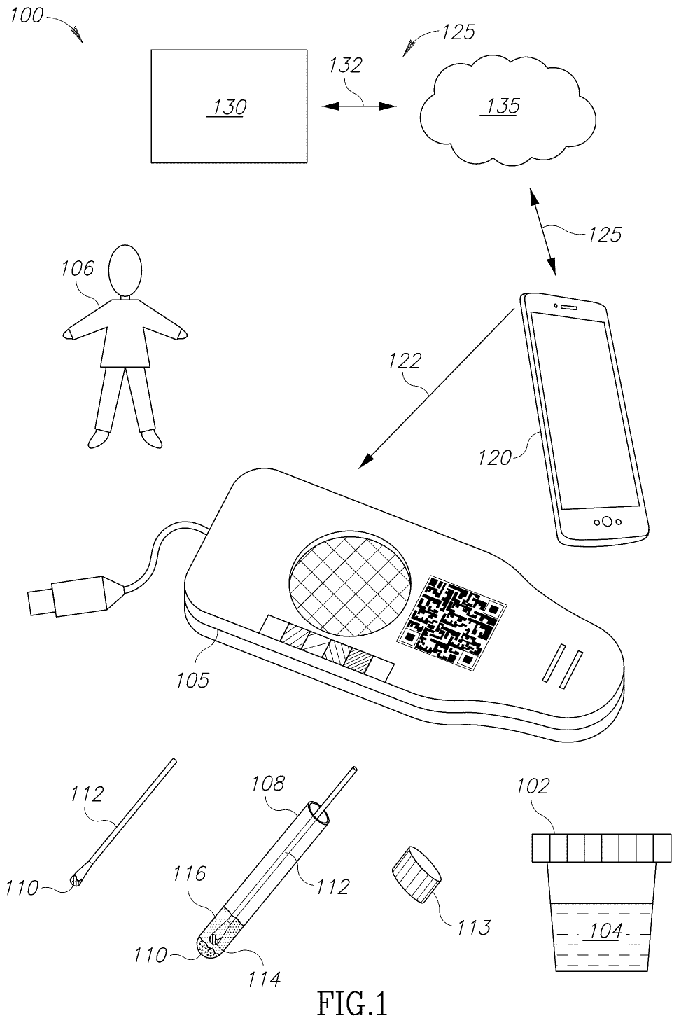

FIG. 1 is a schematic illustration of a system 100 for self-administering a culture test, according to certain exemplary embodiment. System 100 includes a specimen cup 102 for collecting a fluid sample 104 from a subject 106. In some embodiments, the fluid sample 104 can be urine, spit, infection, blood or the like. System 100 includes a swab 112 for collecting a specimen sample 114 from fluid sample 104. System 100 can include a vial 108 storing bacterial nutrition liquid broth 110 to facilitate the growth of the bacteria inserted into bacterial nutrition liquid broth 110. In some embodiments, vial 108 can have a total volume within a range of 2.5-3 milliliters (“ml”) and can store a volume of bacterial nutrition liquid 110 within a range of 0.5-1.5 ml. Swab 112 with the specimen sample 114 is inserted into vial 108 thereby mixing specimen sample 114 with bacterial nutrition liquid broth 110 to produce a specimen and nutrient mix 116. System 100 includes an apparatus 105 for self-administering a culture testing apparatus. Specimen and nutrient mix 116 is poured into apparatus 105 to facilitate monitoring bacterial growth of the bacteria during an incubation period, for example, during a 6-14 hours incubation.

In some embodiments, system 100 includes a smart device 120, such as a smartphone, tablet, camera or the like, configured for recording images, as represented by arrow 122. Smart device records one or more images of apparatus 105 and provides an analysis of image to determine the specific bacteria and which antibiotic medication should be recommended to subject 106. In some embodiments, system 100 can include a server 130 which receives data from smart device 120 via network 135, as represented by arrows 125, 132, to facilitate contacting a medical practitioner or to store the data, such as the images recorded and the analysis of the bacterial growth after the incubation period.

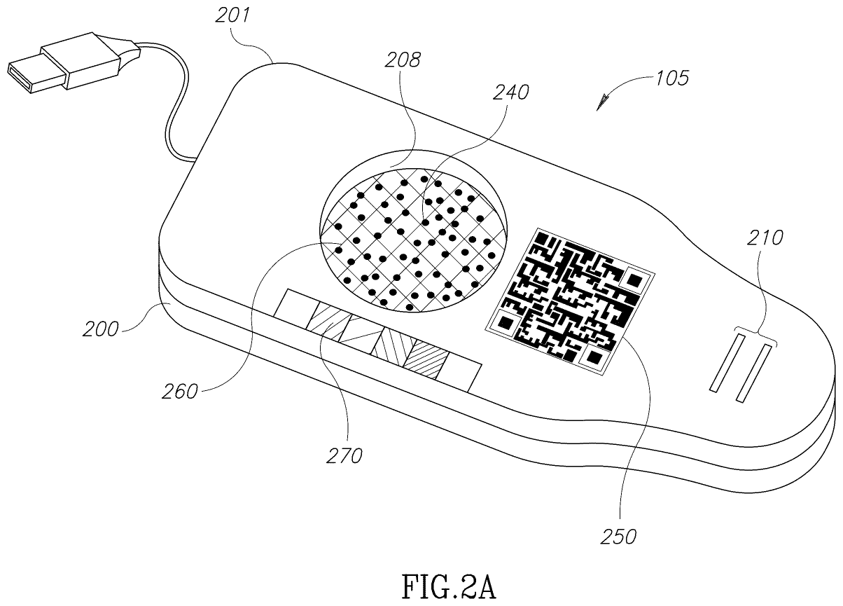

Reference is now made to FIGS. 2A-2B, which are schematic illustrations of apparatus 105 having a housing 200 and a cover 201 for storing and growing the bacteria, according to certain exemplary embodiments. Cover 201 can include ventilation openings 210. In some embodiments, apparatus 105 can include a color scheme 270, or basic colors for calibration of a software configured for determining bacterial growth 260 and the specific bacteria growing on filter 240. Apparatus 105 includes a compartment 208, configured for growing bacteria and exposing the bacteria to antibiotics. In some embodiments, apparatus 105 includes a window cover 231 for covering compartment 208 while enabling visual tracking of the bacterial growth.

Reference is now made to FIG. 2B, which is a side cut through view schematic illustration of apparatus 105, according to certain exemplary embodiments. Apparatus 105 includes a hydrophilic filter 240 or hydrophilic membrane to facilitate bacteria growth on hydrophilic filter 240. In some embodiments, hydrophilic filter 240 has a spacing of 0.45 micrometers to prevent the bacteria from falling off of hydrophilic filter 240. Apparatus 105 includes a pad 250, that can have a width within a range of 1-150 micron, or absorb material or open cell foam, adjacently positioned under the filter to absorb the liquid without the bacteria and thereby supplying the nutrients through the filter 240 for bacterial growth on the hydrophilic filter 240 during incubation. In some embodiments, hydrophilic filter 240 and pad 250 are positioned adjacent to enable the bacteria to easily access the nutrient source.

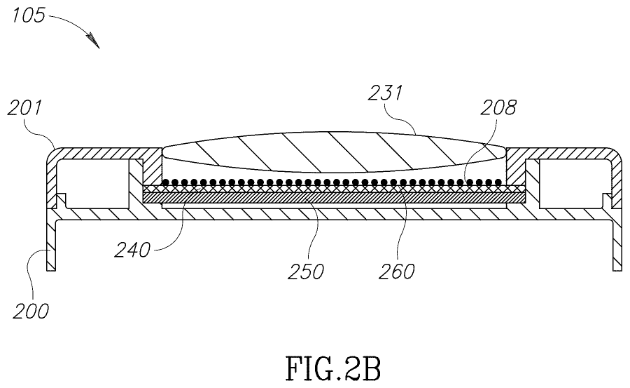

FIG. 3 is a schematic illustration of apparatus 105 including a swinging window cover 300, according to certain exemplary embodiment. Cover 300 includes a cover window 205 connected to a rod 206. Rod is movably affixed between hinges 207, 209 to facilitate swinging cover window 205 onto compartment 208, as represented by arrow 220, thereby sealing compartment 208 during the bacterial incubation period. In some embodiments, cover window 205 is transparent.

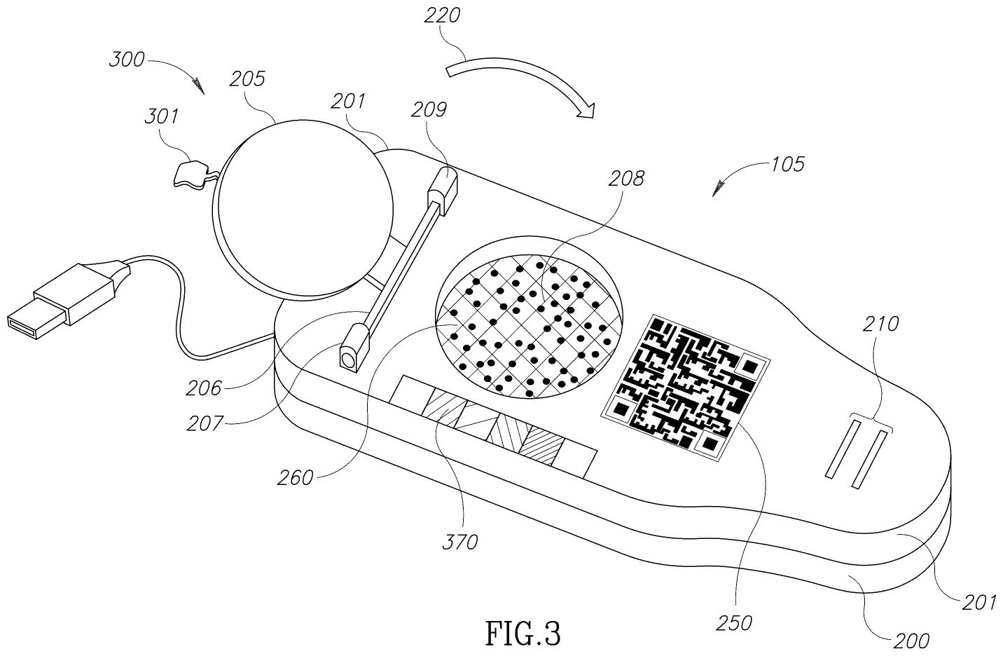

FIGS. 4A-4B are schematic illustrations of apparatus 105 including a slide-on cover 400, according to certain exemplary embodiment. slide-on cover 400 is positioned over compartment 208 as shown in FIG. 4B. slide-on cover 400 includes a handle 410 to facilitate holding sliding cover. In some embodiments, sliding cover can have a clear cover 430 to enable visual monitoring of the bacteria incubation within apparatus 105. Slide-on cover 400 is slid between rails 405, 406 in the direction of arrows 420, 425 to insert and extract, respectively.

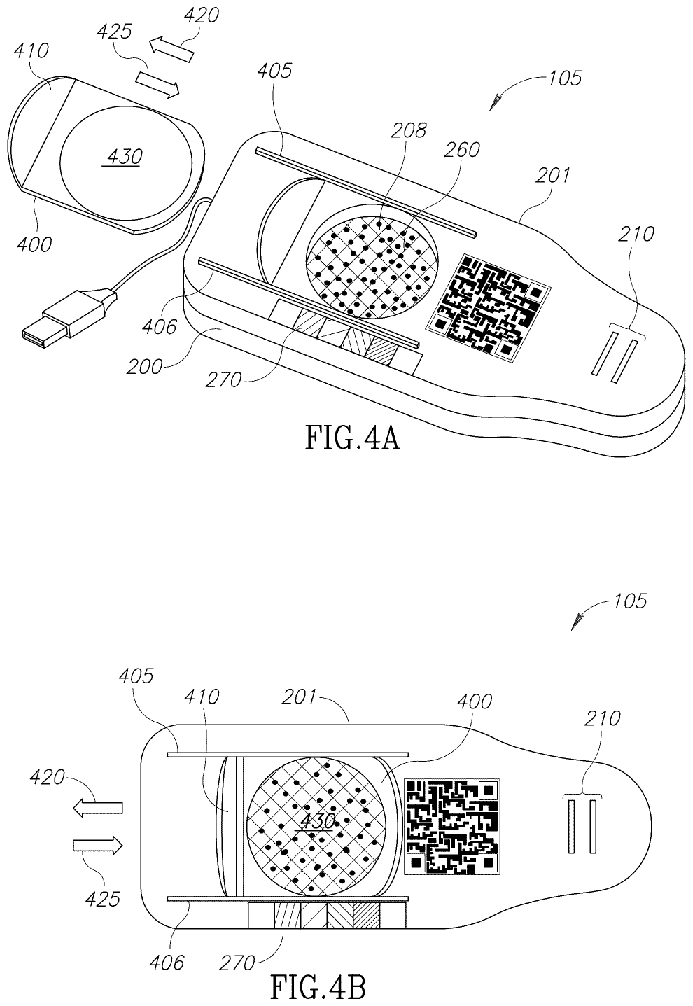

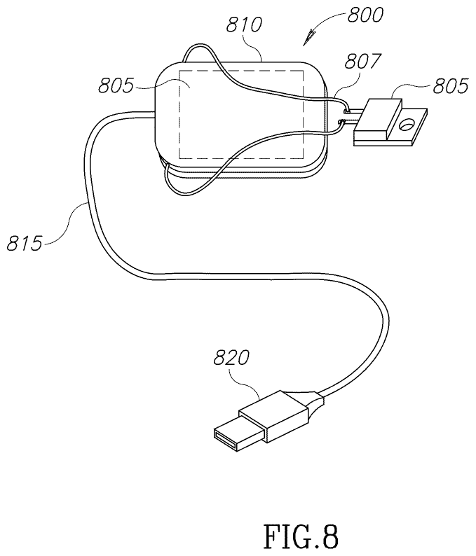

FIG. 5 is a schematic illustration of a housing 200 of apparatus 105, according to certain exemplary embodiment. Housing 200 includes a side port 505 for insertion of a wire 815 (FIG. 8) of electric heating element 805 (FIG. 8) configured to provide an incubation temperature, for example, a temperature with a range of 35-37 Celsius, within housing 200. In some embodiments, a thermostat 809 (FIG. 8), which is a standard closed type, is calibrated to a range of 35-37 Celsius to facilitate moderating the temperature of heating element. The thermostat is connected to the electric circuit of the heating element 805 by electric wires 807.

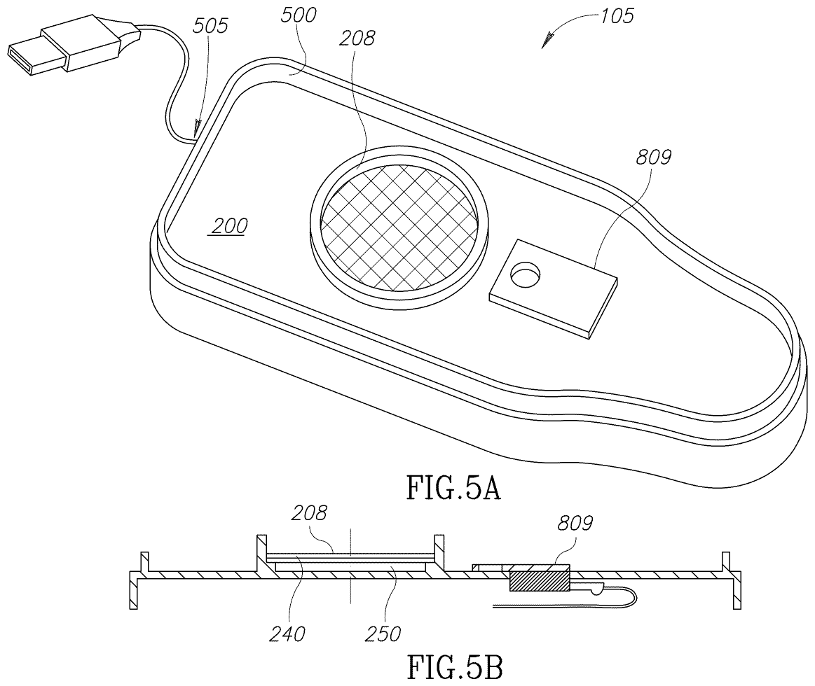

FIG. 6 is a schematic illustration of hydrophilic filter 240, according to certain exemplary embodiment. In some embodiments, filter 240 can be a mesh having a density to facilitate bacteria growth on hydrophilic filter 240. In some embodiments, filter 240 can include a membrane for bacterial growth.

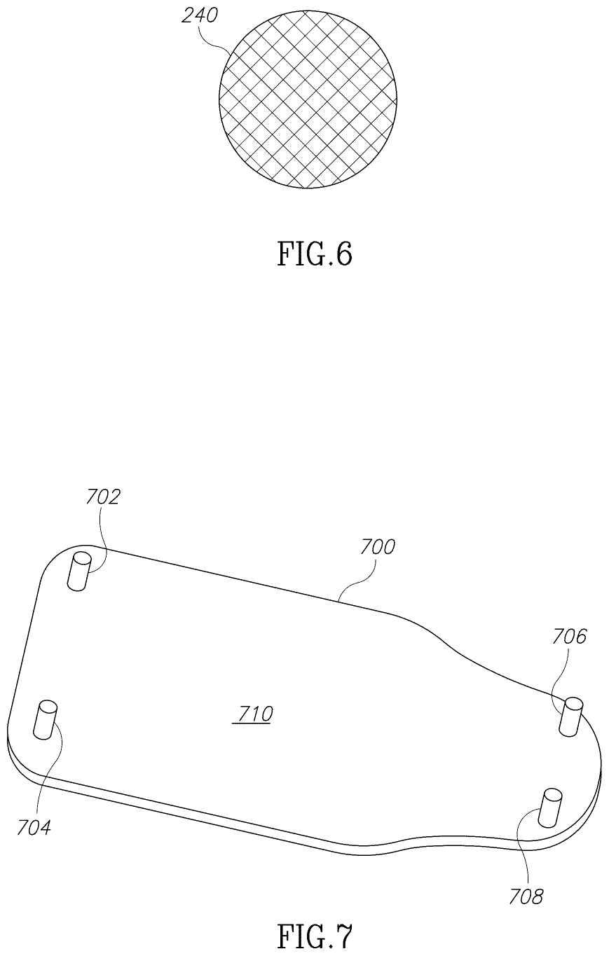

FIG. 7 is a schematic illustration of an internal base 700 of apparatus 105 (FIG. 1), according to certain exemplary embodiment. Internal base 700 seals heating element 805 (FIG. 8) and thermostat 809 (FIG. 8) within housing 200 to facilitate heating an incubation environment. Base 700 can include pins 702, 704, 706, 708 operative to facilitate connecting base 700 to apparatus 105.

FIG. 8 is a schematic illustration of a heating element 800 for apparatus 105 (FIG. 1), according to certain exemplary embodiment. Heating element 800 includes a heating platform 810 which is positioned adjacent to apparatus 105, for example, within housing 200. Heating element 800 includes a power source, such as a USB 820. In some embodiments, heating element 800 includes a 35-37 thermostat 809 for monitoring a temperature of apparatus 105 while it is being heated.

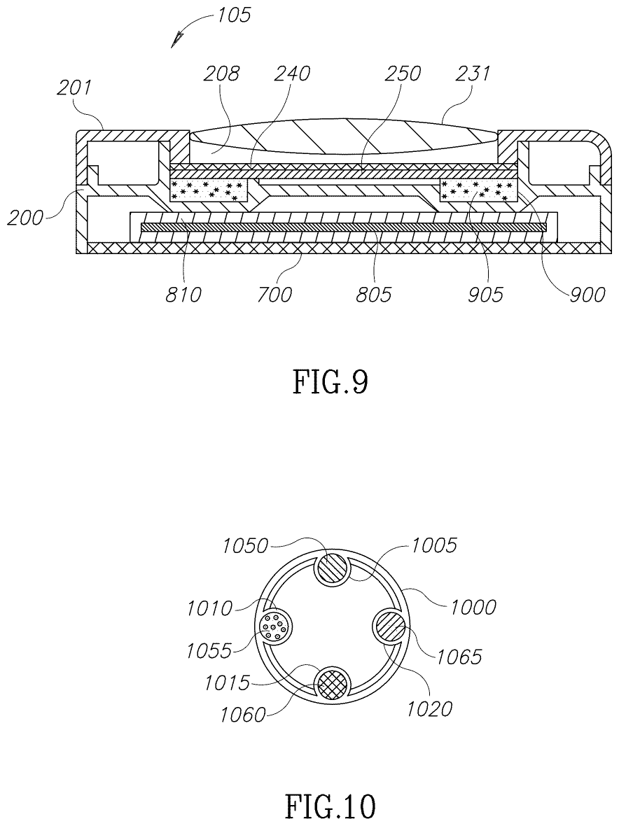

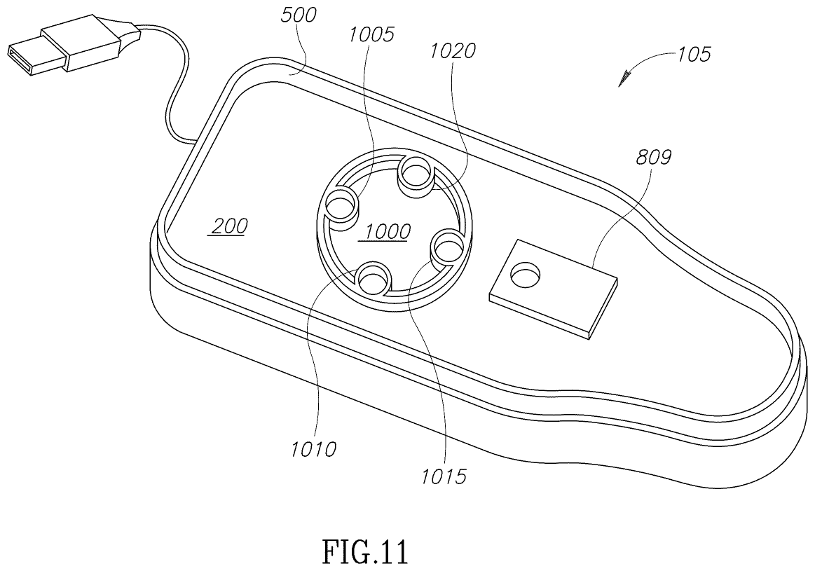

FIG. 9 is a side cutaway view schematic illustration of apparatus 105 having antibiotic compartments 900, according to certain exemplary embodiment. In some embodiments, compartments 900 store dried antibiotics 905 or antibiotic discs 1000 (FIG. 10) to expose it to the bacteria growing on hydrophilic filter 240 during incubation. The bacteria are exposed to the antibiotics to facilitate determining whether the antibiotics 905 are effective for treatment of the bacterial infection.

FIG. 10 is a schematic illustration of an antibiotic compartment 1000 of apparatus 105 (FIG. 1), according to certain exemplary embodiment. Antibiotic disc 1000 or dried antibiotic 1000 is configured to store multiple types of antibiotics in separate compartments. For example, antibiotic disc 1000 includes four compartments 1005, 1010, 1015, 1020, for storing four different types of antibiotics 1050, 1055, 1060, 1065. In some embodiments, Antibiotic disc 1000 is circular to facilitate a maximum exposure of the antibiotics to the bacteria 1050, 1055, 1060, 1065.

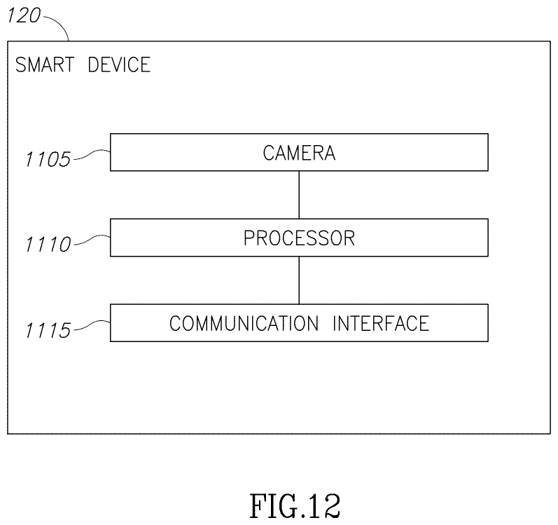

Reference is now made to FIG. 11, which is a schematic illustration of antibiotics disc 1000 positioned within compartment 208 (FIG. 2A), according to certain exemplary embodiments. Antibiotics disc 1000 is positioned to be adjacent to hydrophilic filter 240 or pad 250 to ensure optimal exposure of the bacteria to the antibiotics.

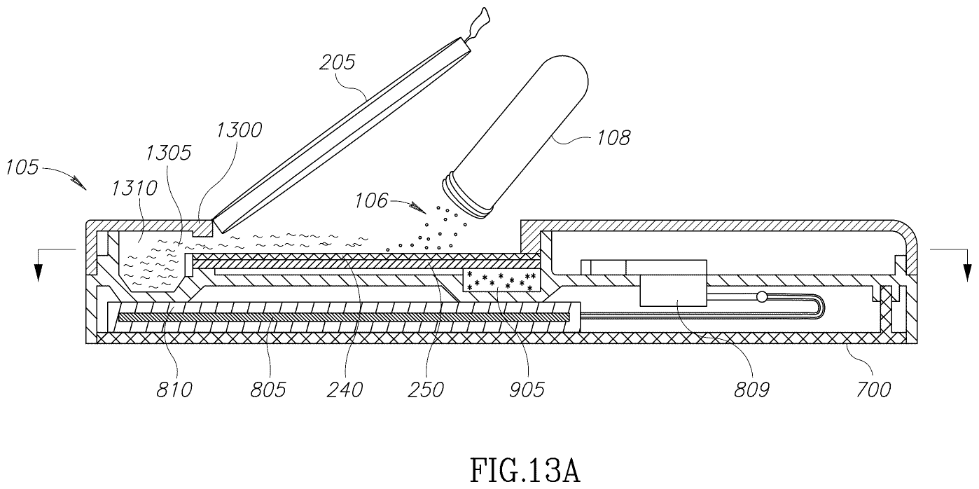

FIG. 12 is a schematic illustration of smart device 120, according to certain exemplary embodiment. Smart device 120 includes a camera 1105 for recording images or video of the growth of the bacteria in apparatus 105 (FIG. 1) after incubation. Smart device 120 includes a processor 1110 configured for analyzing the images recorded by camera 1105 to determine whether there is a bacterial infection 260 (FIG. 2A). In some embodiments, processor 1110 can also determine which antibiotic was the most effective at treatment of the bacteria. Smart device 120 can include a communication interface 1115 configured to communicate with server 130 to provide the analysis and images to a medical practitioner or the like.

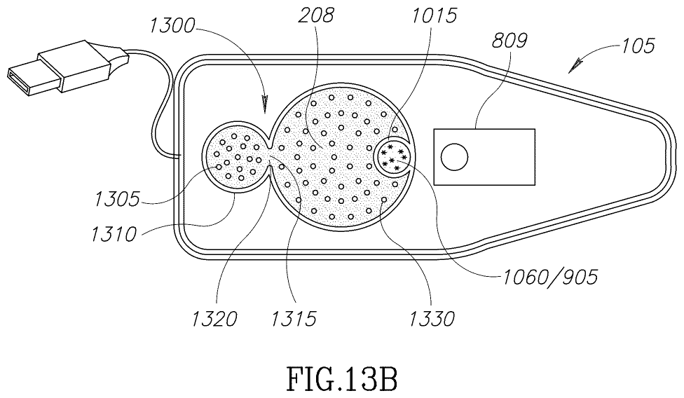

Reference is now made to FIG. 13, which is a schematic illustration of apparatus 105 including a carbon generator 1300 having one or more antibiotics, according to certain exemplary embodiments. Carbon dioxide (“CO2”) generator 1300 includes a predetermined quantity of sodium bicarbonate 1305, for example, withing a range of 0.33-0.5 grams. When sodium bicarbonate 1305 mixes with fluid from the bacterial nutrition liquid broth, the mixture results with a chemical reaction that produces CO2 1330 which travels to the bacteria on the hydrophilic filter 240 (FIG. 2B). In some embodiments, the sodium bicarbonate 1305 can be in a pill form, powder form, or the like. In some embodiments, sodium bicarbonate 1305 can be stored under filter 240 and pad 250. In some embodiments, carbon dioxide generator 1300 can include a tub 1310, which can have a circumference of 12 millimeter (“mm”) and depth of 3 mm, which is adjacent and connected to compartment 208. In certain embodiments, tub 1310 and compartment 208 the under pad 250 are connected with a bottom connection 1315 and a top connection 1320 to facilitate fluid flow from pad 250 to tub 1300 and CO2 1330 flow from tub 1300 to compartment 208 respectively. In some embodiments, cover window 231 hermetically seals compartment 208 to prevent CO2 1330 from escaping from compartment 208.

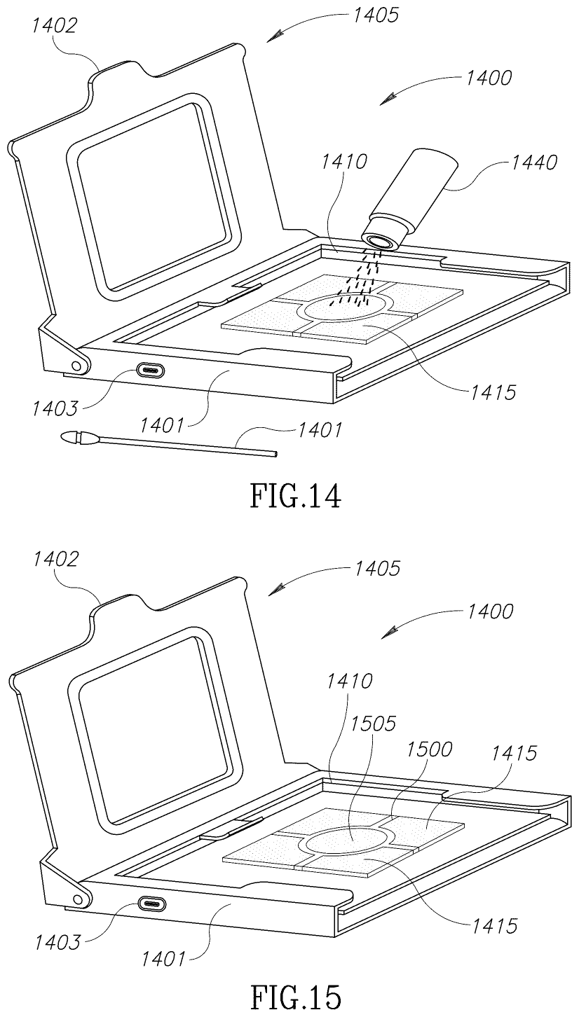

FIG. 14 is a schematic illustration of a second embodiment of a system 1400 for self-administering a culture test, according to certain embodiments. System 1400 includes an apparatus 1405 for self-administering a culture test. Apparatus 1405 includes housing 1401, a transparent cover 1402, and a compartment 1410 for positioning of a pad 1415 on card 1600. Pad 1415 is composed of a dry chromogenic medium, which is coated with a growth medium and a chromogenic substrate. System 1400 includes a swab 1430 and a vial 1440. Swab 1430 is used to collect a fluid sample, such as urine, and mixed with dilution water in a vial 1440. The mixture of urine sample and dilution sterile water or other liquid or broth such Buffered Peptone Water, Butterfield's Buffer, Phosphate Buffer, is poured from vial 1440 onto medium 1415 for incubation.

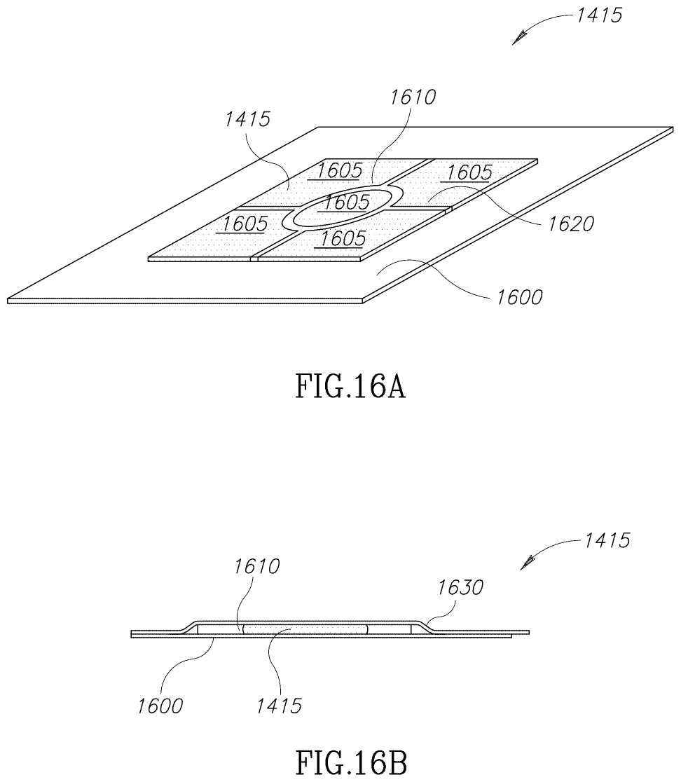

FIG. 15 is a schematic illustration of an apparatus of the system of FIG. 14 for self-administering the culture test, according to certain exemplary embodiments. Apparatus 1405 includes a USB connector 1403 operative to connect apparatus 1405 to a power source and to operate a heating element (not shown) that is operative to expedite incubation of cultures on the medium on pad 1415. Compartment 1410 is operative for positioning pad 1415 and card 1600 therewithin and positioning a partitioning element 1500 on card 1600. Partitioning element 1500 is operative to partition off different regions of the pad 1415. At least one region 1505 is designated as a control region for culture only. The other regions facilitate aspartate Antimicrobial Susceptibility Test (“AST”) testing using different antibiotics.

The sample fluid from the vial is poured into a control region 1505, and the excess liquid overflows into the other regions. The partitioning element 1500 prevents the wet antibiotic from spreading across the entire pad and mixing everything, including into the control region 1505, by containing the distribution and diffusion within the pad 1415.

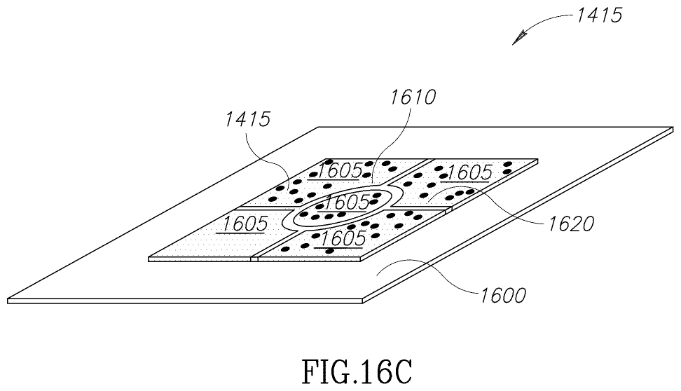

FIGS. 16A-16C are schematic illustrations of pad 1415 and according to certain exemplary embodiments. Pad 1415 includes a lower film 1600 on which medium 1605 is positioned. Pad 1415 includes a partitioning element 1610 configured in a predetermined shape to separate medium 1605 into regions. For example, partitioning element 1610 is cut according to an internal shape of the compartment's surface. Medium 1605 in each region is glued or permanently fixed to film 1600. Partitioning walls 1620 of partitioning element 1610 are composed of a solid material that prevents contamination of the pad in different regions. In some embodiments, the partition walls 1620 are of a thickness within as range of 0.20-0.65 millimeters (“mm”) or they can each have a different width. In some embodiments, the different regions have unified shape or the regions can have different shapes.

FIG. 16B is a schematic cross of the card, contain a lower film 1600, partitioning element 1610, pad 1415 and a upper transparent thin film 1630. The upper film 1630 applicable for storing pad 1415 and card 1600 before using, lift-up for dropping the liquid onto the pad 1415 and re-covering.

FIG. 16C are schematic illustrations of pad 1415 and according to certain exemplary embodiments after incubation and AST

The antibiotic region can be a DISC, a dried antibiotic spot, dried antibiotic within a dried pad, or antibiotic delivered in another way. Accepted values are at the microgram level. The antibiotic can be on pad 1415, beneath it, adjacent to it outside pad 1415, within it, or outside the compartment but connected to it via fluid transfer, or by another method.

In some embodiments, additional regions can be used for additional incubation or control that results in a reduced growth after incubation. In the additional control region, isolated colonies are formed that can be counted and visually analyzed more effectively, without interfering with the test based on dilution in the first compartment, which serves as an example of 1:1,000.

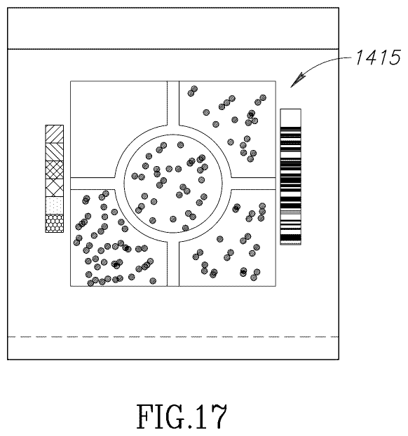

FIG. 17 is a schematic illustrations of pad with different growth cultures after incubation on pad 1415, according to certain exemplary embodiments.

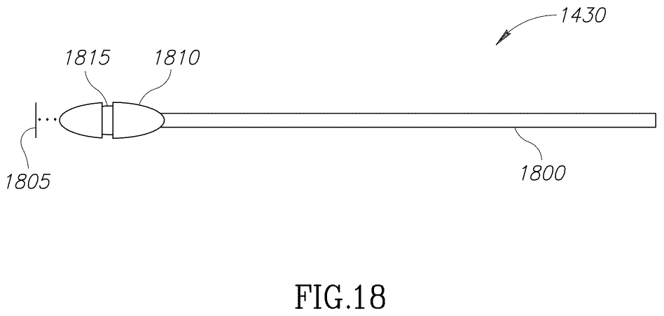

FIG. 18 is a schematic illustration of swab 1430, according to certain exemplary embodiments. Swab 1430 includes a shaft 1800. At a distal end 1805, swab 1430 includes a tip 1810, which includes a peripheral groove 1815 that can collect a predetermined volume of fluid, such as one microliter of fluid. Peripheral groove is operative to collect a sample fluid while the rest of the fluid slides off of tip 1810 and back into urine cup, such as urine cup 102 (FIG. 1).

In the context of some embodiments of the present disclosure, by way of example and without limiting, terms such as ‘operating’ or ‘executing’ imply also capabilities, such as ‘operable’ or ‘executable’, respectively.

Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly including additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry.

The processor and program may constitute the same apparatus, at least partially, such as an array of electronic gates, such as FPGA or ASIC, designed to perform a programmed sequence of operations, optionally including or linked with a processor or other circuitry.

The term computerized apparatus or a computerized system or a similar term denotes an apparatus including one or more processors operable or operating according to one or more programs.

As used herein, without limiting, a module represents a part of a system, such as a part of a program operating or interacting with one or more other parts on the same unit or on a different unit, or an electronic component or assembly for interacting with one or more other components.

As used herein, without limiting, a process represents a collection of operations for achieving a certain objective or an outcome.

As used herein, the term ‘server’ denotes a computerized apparatus providing data and/or operational service or services to one or more other apparatuses.

The term ‘configuring’ and/or ‘adapting’ for an objective, or a variation thereof, implies using at least a software and/or electronic circuit and/or auxiliary apparatus designed and/or implemented and/or operable or operative to achieve the objective.

A device storing and/or including a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which includes one or more executable instructions for implementing the specified logical function(s). It should also be noted that, in some alternative implementations, illustrated or described operations may occur in a different order or in combination or as concurrent operations instead of sequential operations to achieve the same or equivalent effect.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value implies within an inclusive range of −10% to +10% of the respective magnitude or value.

Unless otherwise specified, the terms ‘about’ and/or ‘close’ with respect to a dimension or extent, such as length, implies within an inclusive range of −10% to +10% of the respective dimension or extent.

Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub ranges as well as individual numerical values within and about the boundary of that range. Any numeric value, unless otherwise specified, includes also practical close values enabling an embodiment or a method, and integral values do not exclude fractional values. A sub range values and practical close values should be considered as specifically disclosed values.

As used herein, ellipsis ( . . . ) between two entities or values denotes an inclusive range of entities or values, respectively. For example, A . . . Z implies all the letters from A to Z, inclusively.

Terms in the claims that follow should be interpreted, without limiting, as characterized or described in the specification.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An apparatus for self-administering an infection culture test, the apparatus comprising:
a housing having a cover and a compartment for storing a bacterial culture and to facilitate bacterial growth, wherein the compartment comprises:
a pad having a partitioning element attached to a film, wherein the partitioning element separates a dry chromogenic medium into regions, wherein the medium is coated with a growth medium and a chromogenic substrate; and,
a transparent cover to hermetically seal the compartment.

2. The apparatus according to claim 1, further comprising a heating device, wherein the heating element is attachable to the housing to facilitate providing a predetermined temperature to expedite growth of the bacteria during the predetermined incubation period.

3. The apparatus according to claim 1, further comprising ventilation ports.

4. The apparatus according to claim 1, wherein the housing further comprising an antibiotics compartment to facilitate exposing the bacteria to antibiotics during the predetermined incubation period.

5. The apparatus according to claim 1, further comprising an antibiotics compartment having a plurality of compartments for storing at least one type of antibiotic effective for treatment of the bacteria.

6. The apparatus according to claim 1, further comprising a color scheme and basic colors to facilitate calibrating a camera configured to record at least one image thereby providing a visual comparison for determining whether there is a bacterial infection and to identify the specific bacterial culture.

7. The apparatus according to claim 1, wherein the window cover is connected to the housing with hinges to facilitate opening and closing the cover.

8. The apparatus according to claim 1, wherein the window cover is configured to slide onto the compartment.

9. The apparatus according to claim 1, further comprising a carbon dioxide generator configured to generate carbon dioxide thereby expediting culture growth.

10. A system for self-administering an infection culture test, the system comprising:
apparatus for self-administering the infection culture test, the apparatus comprising:
a housing having a cover and a compartment for storing a bacterial culture and to facilitate bacterial growth, wherein the compartment comprises:
a pad having a partitioning element attached to a film, wherein the partitioning element separates a dry chromogenic medium into regions, wherein the medium is coated with a growth medium and a chromogenic substrate;
a transparent cover to hermetically seal the compartment;
at least one smart device configured to record at least one image of the bacteria within the apparatus and provide a determination whether an infection is present and to identify what specific bacteria is present.

11. The system according to claim 10, wherein the at least one smart device comprises:
at least one camera for recording the at least one image;
at least one processor configured to analyze the at least one image to determine whether an infection is present by identify the specific bacteria; and,
at least one communication interface configured to provide the at least one image and determination of infection to a medical practitioner for assistance.

12. The system according to claim 11, wherein the smart device further comprises a memory for storing the images and determination of infection.

13. The apparatus according to claim 11, wherein the apparatus further comprising a color scheme and basic colors to facilitate calibrating the at least one camera configured to record at least one image thereby providing a visual comparison for determining whether there is a bacterial infection and to identify the specific bacterial culture.

14. The system according to claim 10, further comprising:
a specimen cup for collecting a fluid sample;
a swab for taking a specimen sample from the fluid sample; and,
a vial including a nutrition liquid broth, wherein the specimen sample is inserted into the vial to create a bacterial mixture for placing in the housing.

15. The system according to claim 10, further comprising a heating device, wherein the heating device is attachable to the housing to facilitate providing a predetermined temperature to expedite growth of the bacteria.

16. The system according to claim 10, wherein the apparatus further comprising ventilation ports.

17. The system according to claim 10, wherein the housing further comprising an antibiotics compartment to facilitate exposing the bacteria to antibiotics.

18. The system according to claim 10, wherein the apparatus further comprising an antibiotics compartment having a plurality of compartments for storing at least one type of antibiotic to which the bacteria is exposed while the bacteria is within housing.

19. The system according to claim 10, wherein the window cover is connected to the housing with hinges to facilitate opening and closing the cover.

20. The system according to claim 10, wherein the window cover is configured to slide onto the compartment.
